(19) United States
(12) Patent Application Publication
(10) Pub. No.: US 2003/0212316 A1
(43) Pub. Date: Nov. 13, 2003

(54) Method and apparatus for determining blood parameters and vital signs of a patient

(76) Inventors: Jeffrey M. Leiden, Glencoe, IL (US); Omar S. Khalil, Libertyville, IL (US); Eric Brian Shain, Glencoe, IL (US); Stanislaw Kantor, Buffalo Grove, IL (US); Shu-jen Yeh, Grayslake, IL (US); James J. Koziarz, Highland Park, IL (US); Charles F. Hanna, Libertyville, IL (US); Xiaomao Wu, Gurnee, IL (US); Ronald R. Hohs, Kenosha, WI (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/144,224

(22) Filed: May 10, 2002

(51) Int. Cl. A61B 5/00

(52) U.S. Cl. 600/323

(57) ABSTRACT

A method of monitoring a patient that comprises a non-invasive measurement of the hematocrit value or the concentration of hemoglobin coupled with the measurement of one or more vital signs. These vital signs include, but are not limited to, cardiac pulse rate, blood pressure, and arterial blood oxygenation. The invention also provides an apparatus for monitoring changes in the hematocrit value of a patient, in combination with one or more of the patient's vital signs.

### BACKGROUND OF THE INVENTION

1. Field of the Invention

This invention relates to an apparatus and a method for monitoring the condition of a patient, more particularly, for monitoring the condition of a patient by monitoring the change in a blood parameter, such as the concentration of hemoglobin or the hematocrit value, combined with changes in the patient's vital signs, such as cardiac pulse rate, oxygen saturation, and blood pressure.

2. Discussion of the Art

Measuring the vital signs of a patient is a standard practice in the care of a patient. Vital signs include cardiac pulse rate, temperature, breathing frequency, and blood pressure. Vital signs are usually measured at the physician's office, before the patient is admitted to a hospital, and routinely during hospital care. Additionally, these vital signs are continuously, or at least frequently, monitored during and after a surgical operation. In addition to cardiac pulse rate, temperature, and blood pressure, another parameter, arterial blood oxygen saturation, is monitored during and after a surgical procedure. A decrease in cardiac pulse rate, blood pressure, or blood oxygen saturation is indicative of a deterioration of the condition of the patient.

The cardiac pulse rate is an important vital sign for determining the health status of a patient and for monitoring the patient's status during intensive care and postoperative recovery. A decrease in cardiac pulse rate indicates a decrease in the frequency at which the heart contracts and expands, and thus indicates a decrease in cardiac sufficiency. An irregular cardiac pulse rate is an indication of heart murmur and asynchronous cardiac performance. Monitors that incorporate blood oxygen saturation measurements and cardiac pulse rate are commercially available. A single sensor is used to determine both parameters.

Blood pressure is another important vital sign for determining the health status of a patient and for monitoring the patient's status during intensive care and postoperative recovery. Two values of the blood pressure are monitored, the systolic blood pressure, which is the pressure induced by the contracting heart, and the diastolic blood pressure, which is the ambient pressure in the vascular system as the heart expands. A decrease in blood pressure from the normal level of blood pressure is an indication of a decrease in the capacity of the heart to pump blood, and an increase in blood pressure is an indication of excessive pressure in the blood vessels, which may lead to hemorrhage.

Measurement of blood pressure involves the placement of a pressure cuff around the arm and inflation of the cuff while a stethoscope is placed over the brachial artery in the arm and under the cuff. When the pressure is equal to or higher than the systolic pressure, arterial occlusion occurs, and the stethoscope will detect no pulses. The pressure induced by the cuff is slowly reduced, and the systolic pressure is the value of the pressure at which the cardiac pulse signal is first detected as an audible pulse by the stethoscope. The pressure induced by the cuff is gradually lowered an additional amount, and the diastolic pressure is subsequently determined to be the pressure at which the audible pulse signal vanishes. Automated blood pressure devices that do not require the use of a stethoscope are available. Pressure sensors are used to determine the appearance and disappearance of the cardiac pulse as a function of pressure applied at the cuff. Blood pressure measurements are performed intermittently, on account of the time required to inflate and to deflate the pressure cuff. The blood pressure cuff, with its control, is an independent probe and is not synchronized with the cardiac pulse rate measurement or the arterial blood oxygen measurement.

The hematocrit value indicates the anemic status of a patient. A decrease in the hematocrit value during or after surgery is indicative of internal bleeding. Internal bleeding can eventually lead to a drop in cardiac pulse rate and blood pressure. Concomitant changes in hematocrit value, cardiac pulse rate, and blood pressure, and the magnitude of these changes, will indicate the severity of the bleeding and the urgency of intervention. Timely intervention may allow a patient's life to be saved.

The concentration of hemoglobin and the ratio of oxygenated hemoglobin to total hemoglobin in blood are important parameters for indicating the anemic state and wellness of a patient. Hemoglobin is the protein that transports oxygen. It has a molecular weight of 65,500 Daltons; thus, 1 gram of hemoglobin is equivalent to 1.55×10⁻⁵ mole. The concentration of hemoglobin is expressed in g/dL. The hematocrit value is the ratio of volume of red blood cells to total blood volume, which comprises the volume of red blood cells and the volume of plasma. The hematocrit value is expressed as a percentage (i.e., percentage by volume of red cells in whole blood). While the measurement of concentration of hemoglobin provides an indication of the oxygen transport status of the patient, the measurement of the hematocrit value provides an indication of both red blood cells for transport of oxygen and plasma for transport of nutrients. The measurement of the hematocrit value is particularly important when a change in body hemodynamics is expected, such as during operations of long duration, such as, for example, cardiac and orthopedic surgery. Other applications of the measurement of the hematocrit value include the treatment of hemorrhage in accident victims and the monitoring of cancer patients undergoing chemotherapy. Yet another application of the measurement of the hematocrit value involves monitoring patients undergoing kidney dialysis to reduce the potential for incomplete dialysis or excessive dialysis of the patient. Incomplete dialysis leaves toxins behind. Excessive dialysis leads to shock.

The standard method currently used for measuring the hematocrit value is an invasive method. Typically, a blood sample is obtained from a patient or a donor and centrifuged in a capillary tube to separate red blood cells from plasma. The length of the column in the capillary tube containing red blood cells and the total length of the column in the capillary tube containing both the red blood cells and the plasma are measured, and the ratio of these lengths is the hematocrit value (Hct). See, for example, Morris, M. W., and Davey, F. R., “Basic examination of blood”, in Clinical Diagnosis and Management by Laboratory Methods, Henry, J. B., ed., W. B. Saunders Company, Philadelphia, Pa. (1996), pages 549-559.

Other methods for determining the hematocrit value involve the use of a flow cytometer, wherein a known volume of blood is injected in a fluid stream and the number of red blood cells (RBC) and the mean volume thereof is determined. The total volume of RBC is calculated and the hematocrit value is determined from the volume of the sample and the volume of total RBC. Concentrations of hemoglobin can be determined in vitro by a photometric method, wherein a blood sample is hemolyzed and the heme moiety is released from hemoglobin at a high pH level.

The absorption of this heme moiety is determined at wavelengths of 577 nm and 633 nm.

Methods for the non-invasive determination of the hematocrit value include pulse-based methods and direct current-based methods. Pulse-based methods, such as described by Schmitt et al., “Measurement of blood hematocrit by dual-wavelength near-IR photoplethysmography” SPIE Proceedings 1992; 1641:150-161, exhibit problems in the case of individuals having low peripheral perfusion.

Non-invasive measurement of hematocrit value was recently reported (Wu et al., “Non-invasive determination of hemoglobin and hematocrit using a temperature-controlled localized reflectance tissue photometer” Analytical Biochemistry 2000; 287:284-293, and Zhang et al., “Investigation of noninvasive in vivo blood hematocrit measurement using NIR reflectance spectroscopy and partial least squares regression” Applied Spectroscopy 2000: 54:294-299). Zhang et al. describes a method for determining the hematocrit value in vivo during cardiac bypass surgery. Zhang et al. reported that the temperature of the patient was found to change during surgery. A high number of wavelengths in the near-infrared region of the electromagnetic spectrum and a partial least squares regression analysis were used in an effort to minimize the effect of temperature change on the hematocrit value calculated by this method. Although the device and method described by Zhang et al. provide good calibration and prediction for a given patient during surgery, establishing a model to predict the hematocrit values across more than one patient was less successful. Systematic bias between patients was observed. Part of the observed variations was due to changes in subjects' skin temperature. A method for the determination of concentration of hemoglobin and the hematocrit value is described in WO 01/87151. Steuer et al., U.S. Pat. No. 6,266,546, describes an optical method for the determination of the hematocrit value that uses either the AC or the DC component of the signal at wavelengths of 805 nm and 1300 nm. The possibility of using the same device for determination of oxygen saturation at wavelengths of 660 nm and 805 nm is also disclosed.

Non-invasive monitoring of arterial oxygen saturation by pulse oximetry is a well-established practice in the field of clinical medicine. See Jobsis, “Non-invasive infrared monitoring of cerebral and myocardial oxygen sufficiency and circulatory parameters”, Science 1977; 198:1264-67; Y. Mendelson, “Pulse Oximetry: Theory and applications for noninvasive monitoring”, Clinical Chemistry 1992; 38:1601-1607), and Shiga, et al., “Study of an algorithm based on model experiments and diffusion theory for a portable pulse oximeter”, J Biomed Optics 1997; 2:154-161.

Commercial devices for the noninvasive measurement of arterial oxygen saturation are known as pulse oximeters. A major advantage of pulse oximeters is the ability to provide continuous, safe, and effective monitoring of blood oxygenation at the patient's bedside. Prior to the use of pulse oximeters, arterial oxygen saturation was determined invasively by inserting a catheter in the patient's artery and determining the oxygen content of the blood. In a pulse oximetry measurement, the time variant photoplethysmographic signal, caused by changes in arterial blood volume associated with cardiac contraction is recorded. This signal is attributed to arterial blood components and is sensitive to changes in arterial oxygen saturation. In analyzing pulse oximetry signals, it is assumed that there are no pulses from the surrounding vascular bed and that venous blood does not contribute to the signal.

U.S. Pat. No. 5,101,825 describes a method for determining one or more of the following blood parameters: total hemoglobin, arterial oxygen content, and hematocrit value. The method involves the determination of the change in the mass total hemoglobin (ΔTHb) and the change in blood volume (ΔV) during a cardiac pulse, calculating (ΔTHb/ΔV), and deducing at least one of the aforementioned blood parameters by using a known relationship between the aforementioned blood parameters and the value of (ΔTHb/ΔV).

U.S. Pat. No. 5,964,701 describes a patient monitoring device that is worn by an ambulatory patient. This device has a sensor, which provides a signal based on at least one of skin temperature, blood flow, blood constituent concentration, and cardiac pulse rate of the patient. The device also has a transmitter for transmitting the signal to a receiver for receiving the signal from the finger, and a controller for analyzing the signal. Additional features include an accelerometer to detect motion of the finger and means for determining the location of the patient.

Although there are commercially available devices that measure one or two of these vital parameters, no commercially available device allows the noninvasive measurement of the combination of the hematocrit value together with cardiac pulse rate, a blood pressure parameter, or oxygen saturation in a synchronized manner. Further, there are no commercially available devices that allow such a noninvasive measurement by means of a common probe, which would simplify the measurement and improve the interaction between the patient and the device. In addition, there are no devices available that can measure these parameters simultaneously and continuously. Although several methods and devices are described in the art for the determination of hemoglobin and hematocrit, and several devices are described for the determination of cardiac pulse rate and pulse oximetry, and several devices are used for the determination of blood pressure changes, there is no method or device commercially available for measuring a combination of vital signs and the hematocrit value.

### SUMMARY OF THE INVENTION

This invention provides a method of monitoring a patient that comprises a non-invasive measurement of the hematocrit value or the concentration of hemoglobin coupled with the measurement of one or more vital signs. These vital signs include, but are not limited to, cardiac pulse rate, blood pressure, and arterial blood oxygenation. The invention also provides an apparatus for monitoring changes in the hematocrit value of a patient, in combination with one or more of the patient's vital signs.

The measurement of hematocrit value (or concentration of hemoglobin) gives an indication of the anemic state of the patient. A change in the hematocrit value resulting from of a medical procedure is an indication of internal bleeding. A change in the hematocrit value can also be used to indicate the efficiency of chemotherapy or the action of agents that stimulate the generation of red blood cells.

A change in the hematocrit value can be also used to indicate the efficiency of hemodialysis. Combination of hematocrit measurement with either blood pressure measurement or cardiac pulse rate measurement provides an efficient way to monitor a patient undergoing dialysis to prevent over-dialyzing the patient.

The apparatus and method of this invention can be used in a surgical operating room, in postoperative recovery, in an intensive care unit, in an outpatient surgical facility, in a cardiac catheterization laboratory, in a post-cardiac catheterization recovery unit, in an emergency room and holding area, in a coronary care unit, in a GI endoscopy suite, in a trans-esophageal echocardiography unit, in a renal hemodialysis center, in a routine in-patient hospital bed, in a nursing home, and in a physician's office.

In one aspect, this invention provides an apparatus for monitoring the change in the vital signs and blood parameters of a patient. Vital signs include cardiac pulse rate, arterial oxygen saturation, and blood pressure. The apparatus comprises:

a) means for illuminating a body part of a patient;

b) means for collecting optical signals over a period of time from the body part;

c) means for effecting pressure changes or temperature changes or both of the foregoing types of changes in the body part;

d) means for measuring pressure changes or temperature changes or both types of the foregoing changes experienced by the body part;

e) means for calculating at least one value of at least one blood parameter of the patient from the collected optical signals, the blood parameter including, but not limited to, the concentration of hemoglobin or the hematocrit value;

f) means for determining at least one value of at least one vital sign of the patient from the collected optical signals, the at least one vital sign including, but not limited to, the cardiac pulse rate, blood oxygen saturation, blood pressure, and temperature;

g) means for reporting the value of the at least one blood parameter and the at least one value of the at least one vital sign; and

h) means for providing an alarm, e.g., to a health care giver or a patient monitoring station, when (1) the at least one value of the at least one vital sign crosses a specified cut-off value or (2) the rate of change in the at least one value of the at least one vital sign crosses a specified cut-off value or (3) the at least one value of the at least one blood parameter crosses a specified cut-off value or (4) the rate of change in the at least one value of the at least one blood parameter crosses a specified cut-off value.

In another aspect, this invention provides a method for monitoring a change in the value of at least one vital sign and a change in the value of at least one blood parameter of a patient. The method includes collecting a set of optical measurements and a time domain analysis of the optical measurements. The method comprises the steps of:

a) collecting a set of optical measurements on a body part of a patient over a period of time;

b) determining at least one value of at least one blood parameter of the patient from the set of optical measurements, the blood parameter including, but not limited to, the concentration of hemoglobin or the hematocrit value of the patient;

c) determining at least one value of at least one vital sign of the patient from the set of optical measurements, the at least one vital sign including, but not limited to the cardiac pulse rate, blood pressure, and blood oxygen saturation;

d) reporting a combination of the at least one value of the at least one blood parameter and the at least one value of the at least one vital sign;

e) repeating steps a), b), c), and d) a sufficient number of times until a trend can be observed; and

f) activating an alarm when (1) the at least one value of the at least one vital sign crosses a specified cut-off value or (2) the rate of change in the at least one value of the at least one vital sign crosses a specified cut-off value or (3) the at least one value of the at least one blood parameter crosses a specified cut-off value or (4) the rate of change in the at least one value of the at least one blood parameter crosses a specified cut-off value.

Although a collection of instruments and a collection of non-integrated sensors are available to monitor some of these physiological parameters and vital signs, no single device is capable of measuring the change in hematocrit value, cardiac pulse rate, patient blood pressure, and arterial oxygen saturation. An integrated device for the determination of a plurality of parameters offers ease of manipulation in the operating and recovery rooms, decreases the number of leads and cables attached to the patient, and simplifies monitoring the condition of the patient, interpreting the results, and responding to changes in the patient's condition.

The device and method of this invention offer distinct advantages for care of patients as compared with devices and methods of the prior art. These advantages include the potential for continuous monitoring of a patient. The device of this invention integrates devices for measuring a plurality of vital signs and hematocrit values. When the hematocrit value or the concentration of hemoglobin is combined with cardiac pulse rate and blood pressure, a complete diagnostic picture of the patient's status can be provided.

### BRIEF DESCRIPTION OF THE DRAWINGS

FIG. 1 is a block diagram that describes the apparatus of this invention.

FIG. 2A is a perspective view of the component of the patient-interface module of the apparatus of this invention that contains the optical probe.

FIG. 2B is side view in elevation of a cross-section of the apparatus shown in FIG. 2A.

FIG. 2C is a perspective view, greatly enlarged, of the optical probe of the apparatus shown in FIG. 2A.

FIG. 3 is a flowchart depicting the steps for the determination of hematocrit and vital signs according to the method of this invention.

FIG. 4A shows the effect of a venous occlusion (130 mm Hg) on the change in optical signals, measured at a distance of 1.86 mm from the source of light. The source of light had wavelengths of 660 nm, 735 nm, 810 nm, and 890 nm.

FIG. 4B shows the effect of total occlusion (170 mm Hg) on the change in optical signals, measured at a distance of 1.86 mm from the source of light. The source of light had wavelengths of 660 nm, 735 nm, 810 nm, and 890 nm.

FIG. 5A is a graph showing the intensity of the reflected light from the forearm of a human subject and at a sampling distance of 1.86 mm as a function of time. The temperature of the skin was maintained at 41° C. The source of light had a wavelength of 590 nm. Signals were collected over a period of three minutes.

FIG. 5B is a graph showing an expanded portion of FIG. 5A, the portion extending from the 100-second point to the 150-second point.

FIG. 5C is a plot of the calculated Fourier Transform of the amplitude of the reflected light signal displayed in FIG. 5A.

FIG. 6A shows the optical signal collected at 1.86 mm from the light introduction site, recorded over a 10-second period. The pulse is superimposed on the low-frequency vasomotion and breathing frequency. Noise spikes are also noticeable.

FIG. 6B shows the same signal as shown in FIG. 6A, digitally filtered to eliminate the long-term motions and the noise spikes.

FIG. 6C shows the digitally filtered signal of FIG. 6B, but normalized by dividing the signal by the mean amplitude of the pulses.

FIG. 6D shows the identification of peaks and vallys for calculating the cardiac pulse rate.

FIG. 7A shows a plot of the calculated change of concentration of oxygenated hemoglobin, after a venous occlusion (130 mmHg) (upper trace) and release of pressure, and a total occlusion (170 mm Hg) (lower trace) of a human finger and release of pressure. Temperature of the skin was maintained at 38° C.

FIG. 7B shows a plot of the calculated change of concentration of deoxygenated hemoglobin, after a venous occlusion (130 mmHg) (upper trace) and release of pressure, and a total occlusion (170 mm Hg) (lower trace) of a human finger and release of pressure. Temperature of the skin was maintained at 38° C.

FIG. 7C shows a plot of the calculated change of concentration of total hemoglobin, after a venous occlusion (130 mmHg) (upper trace) and release of pressure, and a total occlusion (170 mm Hg) (lower trace) of a human finger and release of pressure. Temperature of the skin was maintained at 38° C.

FIG. 8A shows a plot of the optical signal as a function of time as cuff pressure is varied from 200 mm Hg to 50 mm Hg.

FIG. 8B shows a plot of the cuff pressure as a function of time as cuff pressure is varied from 200 mm Hg to 50 mm Hg.

### DETAILED DESCRIPTION OF THE INVENTION

The expression “blood flow” means the velocity of red blood cells in vessels. Blood flow is usually determined by means of laser Doppler flowmetry. The expression “blood flux” or “blood perfusion” refers to the movement of red blood cells in vessels as expressed in mass per unit time per specified mass of tissue. Blood flux equals the number of moving red blood cells multiplied by the mean velocity of red blood cells in tissue. Blood flux is also determined by means of laser Doppler flowmetry.

The expression “vasoconstriction” refers to the constriction of the blood or lymph vessels, such as, for example, by the action of a nerve. A chemical agent s such as glucose, or a physical change, e.g., lowering tissue temperature, can induce vasoconstriction. The expression “vasodilatation” refers to the increase in diameter of a blood or lymph vessel, such as, for example, by the action of a nerve. A chemical agent such as insulin, or a physical change, e.g., increasing tissue temperature, can induce vasodilatation. The expression “microcirculation” refers to the movement of blood in blood vessels, e.g., capillaries, arterioles, and venules, as a result of constriction and relaxation of vessel walls. The expression “vasomotion” refers to the rythmic contraction exhibited by the small arteries and arterioles. Vasomotion is reported to be impaired in diabetic subjects, as compared with healthy subjects.

The term “artery” means a blood vessel that conducts blood from the heart to tissues and organs. Arteries are aligned with smooth flat cells (endothelium) and are surrounded by thick muscular elastic walls containing fibrous tissue. Arteries branch repeatedly until their diameter is less than 300 micrometers; these small-branched arteries are called “arterioles”. Arteriole walls are formed from smooth muscle. The function of arterioles is to control blood supply to the capillaries. The term “capillary” refers to a minute hair-like tube (5-20 micrometers in diameter) having a wall consisting of a single layer of flaftened cells (endothelium). Capillary walls are permeable to water, oxygen, glucose, amino acids, carbon dioxide, and inorganic ions. The capillaries form a network in all tissues. Capillaries are supplied with oxygenated blood by the arterioles and pass deoxygenated blood to the venules.

A “vein” is a blood vessel that conducts blood from the tissues and organs back to the heart; the vein is lined with smooth flat cells (endothelium) and surrounded by muscular and fibrous tissue. Walls of veins are thin relative to those of arteries. Diameters of veins are large relative to those of arteries. The vein contains valves that allow unidirectional flow of blood to the heart. A “venule” is a small vein that collects blood from capillaries and joins other venules to form a vein. A venule has more connective tissue than does a capillary, but is permeable to those similar small molecules that are able to permeate capillaries.

Arterioles and venules are connected to the capillary loop via the shunts. The expression “shunt” refers to a passage or a connection (anastomosis) between two blood vessels. An arteriovenous shunt allows passage of blood from an artery (or arteriole) to a vein (or venule) without going through the capillary loop.

The expression “plexus” refers to a braid of blood vessels. In the skin, the “upper plexus” or the “superficial plexus” refers to the braid of arterioles and venules found at the top (shallower) layer of the dermis. The term “lower plexus” or deep plexus” refers to the braid of arterioles and venules found at the lower (deeper) layers of the dermis. Each of the braids is referred to as a “vascular plexus” and both are interconnected.

Arterioles, venules, capillary loops, the upper plexus, and the lower plexus comprise the microvasculature system and are responsible for controlling skin temperature and the flow of blood and nutrients to the skin and disposal of metabolic products from the skin.

The expression “vital sign measurement” refers to a measurement of a basic function of the body. The four main vital signs routinely monitored by medical professionals include: body temperature, cardiac pulse rate, respiration rate (rate of breathing), and blood pressure. Blood oxygenation is not considered a vital sign, but is often measured along with the vital signs. Vital signs are useful in detecting or monitoring medical problems and can be measured in a medical setting, at home, at the site of a medical emergency, or elsewhere.

The expression “cardiac pulse rate” refers to a measurement of the number of times the heart pulses per minute. As the heart pushes blood through the arteries, the arteries expand and contract with the flow of the blood. Taking a pulse not only measures the average rate at which the heart pumps blood into the arteries, but also can indicate heart rhythm (regularity of the pulses) and strength of the pulse. The normal cardiac pulse rate for healthy adults ranges from 60 to 100 pulses per minute (1 Hz-1.66 Hz). The cardiac pulse rate may fluctuate and increase with exercise, illness, injury, and emotions.

The expression “respiration rate” refers to the number of breaths a person takes per minute. The rate is usually measured when a person is at rest and simply involves counting the number of breaths for one minute or counting how many times the chest rises in one minute. Respiration rates may increase with fever, illness, and with other medical conditions. Normal respiration rates for an adult person at rest range from 15 to 20 breaths per minute (0.25-0.3 Hz). Resting respiration rates over 25 breaths per minute (0.4 Hz) or under 12 breaths per minute (0.2 Hz) may be considered abnormal.

The expression “blood pressure” refers to the force of the blood pushing against the artery walls. Every time the heart contracts, it pumps blood into the arteries, resulting in the highest blood pressure limit, which is the systolic blood pressure. Two numbers are recorded when measuring blood pressure. The “systolic blood pressure” refers to the pressure inside the artery when the heart contracts and pumps blood through the body. The “diastolic blood pressure” refers to the pressure inside the artery when the heart is at rest and is filling with blood. Both the systolic and diastolic pressures are recorded in “mm Hg” (millimeters of mercury). High blood pressure, or hypertension, directly increases the risk of coronary heart disease (heart attack) and stroke (resulting from hemorrhage or formation of a blood clot in a blood vessel of the brain). When blood pressure is high, the arteries may increase the resistance to the flow of blood, thereby requiring the heart to exert greater force, i.e. to pump harder in order to push the blood into the arteries to circulate the blood. According to the American Heart Association, high blood pressure for adults is defined as systolic pressure of 140 mm Hg or greater and/or diastolic pressure of 90 mm Hg or greater. A single elevated blood pressure measurement is not necessarily an indication of hypertension. Multiple blood pressure measurements over several days or weeks are needed before a diagnosis of hypertension (high blood pressure) can be confirmed.

The expression “arterial oxygen saturation” refers to the oxygenated portion of arterial blood expressed as a percent. The percent oxygen saturation is equal to the ratio of the concentration of oxygenated hemoglobin to the concentration of total hemoglobin, expressed as a percentage. Hemoglobin exists in two forms—oxygenated hemoglobin and deoxygenated hemoglobin. Blood is considered to contain 25% deoxygenated hemoglobin and 75% oxygenated hemoglobin. The sum of the concentrations of the two forms is the concentration of total hemoglobin. In a normal level of arterial oxygen saturation, the arterial blood typically contains about 95% oxygenated hemoglobin. A drop in arterial oxygen saturation to a level below about 90% is indicative of a level of blood oxygen likely to lead to brain damage. Noninvasive monitoring of arterial oxygen saturation by “pulse oximetry” is a well-established practice in the field of clinical medicine. The expression “pulse-oximetry” refers to a technique for measuring arterial oxygen saturation by monitoring the optical signal associated with the cardiac pulse at red and near infrared wavelengths.

Photoplethysmography is an optical measurement of the change in arterial blood volume resulting from cardiac contraction, and a “photoplethysmographic signal” refers to a measured optical signal that is associated with the change in arterial blood volume resulting from cardiac contraction.

In a pulse-oximetry measurement, the time variant photoplethysmographic signal, which is caused by changes in arterial blood volume associated with cardiac contraction, is recorded. This signal is attributed to arterial blood components and is sensitive to changes in arterial oxygen saturation. In analyzing pulse oximetry signals, it is assumed that there are no pulses from surrounding vascular bed and that venous blood does not contribute to the signal.

The “Fourier Transform” is a mathematical expression that decomposes a periodic event into a series of sine waves and cosine waves. The Fourier Transform is used as a method of separating a periodic signal from random noise. Two Fourier parameters are usually calculated, namely the “frequency” and the “amplitude”. The expression “frequency” refers to the the number of periodic oscillations per second and has the unit of Hertz (Hz). One oscillation per second is equivalent to a frequency of 1 Hz. The expression “amplitude” is the sum of the squares of the coefficients in the Fourier Transform equation and is indicative of the magnitude of the oscillations.

Body temperature is one of the vital signs that has clinical importance in assessment of the health status of a patient and in monitoring of a patient. An increase in the body temperature of a patient is indicative of an infection, while a decrease in the body temperature of a patient may indicate shock. A decrease in the temperature of a body part may also indicate improper circulation. The normal body temperature of a person varies depending on gender, recent activity, consumption of food and fluid, time of day, and, in women, the stage of the menstrual cycle. Normal body temperature ranges from 97.8° F. (36.5° C.) to 99° F. (37.2° C.). Body temperature may be abnormal due to fever (high temperature) or hypothermia (low temperature). A fever is indicated when body temperature rises above 98.6° F. (37° C.) orally or 99.8° F. (37.3° C.) rectally. Hypothermia is defined as a drop in body temperature to below 95° F. (35° C.).

The invention involves a method and an apparatus for monitoring changes in the hematocrit value of a patient in combination with one or more vital signs. Vital signs include, but are not limited to, cardiac pulse rate, arterial oxygen saturation, and blood pressure. The method and apparatus can be used in a surgical operating room, in a postoperative recovery unit, intensive care unit, in an outpatient surgical facility, in a cardiac catheterization laboratory, in a post cardiac catheterization recovery unit, in an emergency room and holding area, in a coronary care unit, in a gastrointestinal-endoscopy suite, in a trans-esophageal echocardiography unit, or in a renal hemodialysis center. A change in the hematocrit value following a medical procedure is an indication of internal bleeding. A change in the hematocrit value can be also used to indicate the effectiveness of chemotherapy (increase of the hematocrit value) or the action of agents that stimulate the generation of red blood cells (change in amount of red blood cells, i.e. change in the hematocrit value). A change in the hematocrit value can be also used to indicate the effectiveness of hemodialysis (increase in the hematocrit value without a decrease in blood pressure). Combination of the use of the hematocrit value with either blood pressure or cardiac pulse rate provides a more effective way of monitoring dialysis patients than does measuring one parameter only. Monitoring the change in the hematocrit value and blood pressure during kidney dialysis will prevent over-dialysing the patient. Over-dialysis of a patient will lead to an unnecessary increase in the hematocrit value, which, in turn, may increase blood viscosity. Over-dialysis can also lead to a drop in blood pressure, which may cause fainting.

This invention provides an apparatus for monitoring the vital signs and the blood parameters of a patient. The apparatus comprises:

a) means for illuminating a body part of a patient;

b) means for collecting optical signals over a period of time from the body part;

c) means for effecting pressure changes or temperature changes or both of the foregoing types of changes in the body part;

d) means for measuring pressure changes or temperature changes or both types of the foregoing changes experienced by the body part;

e) means for calculating at least one value of at least one blood parameter of the patient from the collected optical signals, the blood parameter including, but not limited to, the concentration of hemoglobin or the hematocrit value;

f) means for determining at least one value of at least one vital sign of the patient from the collected optical signals, the at least one vital sign including, but not limited to, the cardiac pulse rate, blood oxygen saturation, blood pressure, and temperature;

g) means for reporting the value of the at least one blood parameter and the at least one value of the at least one vital sign; and

h) means for providing an alarm, e.g., to a health care giver or a patient monitoring station, when (1) the at least one value of the at least one vital sign crosses a specified cut-off value or (2) the rate of change in the at least one value of the at least one vital sign crosses a specified cut-off value or (3) the at least one value of the at least one blood parameter crosses a specified cut-off value or (4) the rate of change in the at least one value of the at least one blood parameter crosses a specified cut-off value.

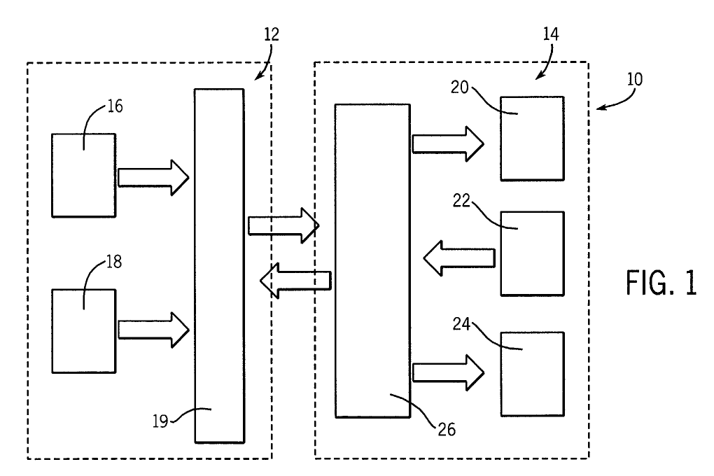

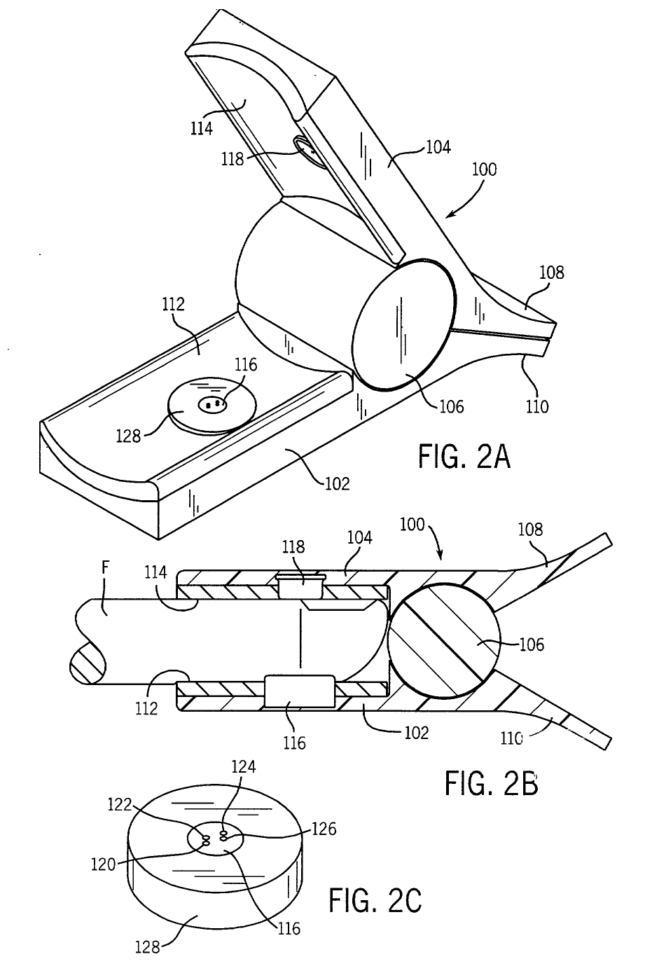

FIG. 1 is a block diagram of an apparatus for carrying out the method of this invention. The components of the block diagram set forth the functions performed by the apparatus 10. The apparatus 10 comprises a patient interface module 12 and a control module 14. The patient interface module 12 comprises a pressure application module 16, an optical measurement module 18, and a plug-in bay 19. The patient interface module 12 has the function of providing points of contact of the pressure application module 16 and the optical measurement module 18 with a body part to obtain measurements of vital signs and optical signals. The control module 14 comprises a computational module 20, an alarm module 22, a communication module 24, and a plug-in bay 26. The control module 14 has the function of providing power and control signals to pressure application module 16 and the optical measurement module 18, pressure control elements, and temperature control elements and receiving signals collected from the optical measurement module 18. The pressure application module 16 performs the function of applying pressure of varying magnitudes to a body part to induce measurable changes in optical signals. A representative example of a pressure application module is an inflatable cuff that can be applied to an arm or a finger. The optical measurement module 18 is an integrated structure comprising at least one optical sensor. An embodiment of an optical sensor is shown in FIGS. 2A, 2B, and 2C and described later. The at least one optical sensor is capable of performing optical measurements of tissue, which measurements are used to calculate the concentration of hemoglobin, the hematocrit value, cardiac pulse rate, blood pressure, and other vital signs. The optical sensors in the optical measurement module 18 can also monitor changes in the hematocrit value and vital signs for patients who are at high risk of having postoperative complications. The pressure application module 16 and the optical measurement module 18 are supplied power through the plug-in bay 19 and are interconnected by means of the plug-in bay 19. The computational module 20 performs the function of performing calculations to compute the concentration of components of the blood and the values of the vital signs. A representative example of the computational module 20 is a personal computer or electronic boards that have microprocessors along with means having the ability to store data in electronic form and the means for communicating that data to other computational devices. The alarm module 22 performs the function of attracting the attention of a nurse or physician or other health care giver to changes in the patient's health status. Representative examples of the alarm module 22 include, but are not limited to, an audible signal or a blinking light. The communication module 24 performs the function of communicating data from the patient from the control module 14 to a nurse's station or a physician's office or to the location of some other health care giver. Representative examples of the communication module 24, include, but are not limited to, a wired connection, a fiber optic connection, or a wireless connection. The computational module 20, the alarm module 22, and the communication module 24 are supplied power through the plug-in bay 26 and are interconnected by means of the plug-in bay 26. The plug-in bay 19 and the plug-in bay 26 are also interconnected.

A representative embodiment of the apparatus of this invention is illustrated in FIGS. 2A, 2B, and 2C. The apparatus 100 is in the form of a clamp that is capable of surrounding and securely attaching to a finger, designated in FIG. 2B by the letter “F”. The lower part 102 of the apparatus 100 and the upper part 104 of the apparatus 100 are connected by a hinge 106. Handles 108 and 110 can be used to move the lower part 102 of the apparatus 100 toward the upper part 104 of the apparatus 100 or to move the lower part 102 of the apparatus 100 away from the upper part 104 of the apparatus 100. It is preferred that both the interior surface 112 of the lower part 102 of the apparatus 100 and the interior surface 114 of the upper part 104 of the apparatus 100 be concave to easily accommodate a finger. An optical probe 116 is fixed onto the interior surface 112 of the lower part 102 of the apparatus 100. The optical probe 116 is substantially similar to the optical probe described in WO 99/59464, incorporated herein by reference. It is preferred that the lower part 102 of the apparatus 100 be biased toward the upper part 104 of the apparatus 100 by a biasing means (not shown) so that contact between the finger and the optical probe 116 can be securely maintained as optical measurements are performed. A biasing means suitable for this purpose is a spring or a strip of elastic material. A detector 118 for detecting light transmitted through the finger and detection electronics (not shown) are fixed onto the interior surface 114 of the upper part 104 of the apparatus 100.

The optical probe 116 comprises a light introduction fiber 120 for introducing light to the finger from a source of light (not shown). The source of light must be capable of generating light at at least two wavelengths. Light that is suitable for use in the apparatus 100 of this invention has wavelengths ranging from about 500 nm to about 2000 nm, inclusive. Light is introduced into the tissue of the finger, and light reflected from the tissue of the finger is collected by a plurality of light collection fibers 122, 124, and 126. Each of the light collection fibers 122, 124, and 126 is positioned at a specified distance from the light introduction fiber 120. The light introduction fiber 120 is connected to the source of light, which is preferably housed in the lower part 102 of the apparatus 100. The light collection fibers 122, 124, and 126 are connected to a set of detectors, amplifiers, and a signal processing board, all of which are also preferably housed in the lower part 102 of the apparatus 100. The set of detectors, amplifiers, and signal processing boards can be housed at a location remote from the apparatus 100. The power input to the apparatus 100 and the signal put out by the apparatus 100 are routed through cables (not shown) to the control unit (not shown). The light introduction fiber 120 and the light collection fibers 122, 124, and 126, sources of light, and detectors housed in the lower part 102 of the apparatus 100 are used to perform optical measurements to obtain data needed to calculate, in a continuous manner, the oxygenation status of blood in the tissue of the finger, the concentration of the different components of hemoglobin, and the change in the concentration of hemoglobin. The components of hemoglobin are oxygenated hemoglobin (HbO₂), deoxygenated hemoglobin (RHb), and total hemoglobin, which is the sum of the oxygenated hemoglobin and deoxygenated hemoglobin. Other parameters, such as oxygen consumption in the tissue, can also be calculated from the data collected by means of the optical probe 116. The hematocrit value can be calculated from the measured concentration of hemoglobin or the change in concentration of hemoglobin by a commonly used multiplication factor.

In a preferred embodiment of this invention, the optical probe 116 is set in a metal disc 128, the temperature of which can be controlled, to allow optical measurements to be carried out at different cutaneous temperatures. The optical probe 116 will sample tissue layers to a depth of approximately 2 mm, when the separation between the light introduction fiber 120 and one of the light collection fibers 122, 124, and 126 is approximately two mm.

In an alternative embodiment, not shown herein, light can be introduced by means of a plurality of light introduction fibers and collected by a single light collection fiber. Such an alternative embodiment is described in WO 99/59464, incorporated herein by reference.

The upper part 104 of the apparatus 100 has a single detector 118, such as a silicon photodiode, for the measurement of light transmitted through the finger. Light transmitted through the finger can be used to calculate arterial oxygen saturation and the cardiac pulse rate. While the optical probe 116 will sample tissue layers to a depth of approximately 2 mm, the signal collected and detected in a transmission mode will have passed through the entire vascular bed of the finger, and thus, will have a larger change in magnitude upon change in blood volume during the pulse than would be expected in the reflectance mode. The same source of light as is used for reflectance measurements can be used for measurement of transmitted light. Measurements in the reflectance mode and measurements in the transmission mode can be carried out simultaneously, if desired.

The apparatus 100 of this invention can be used to monitor fast periodic actions, such as the cardiac pulse, and slow periodic actions, such as breathing rate and the periodic motion resulting from the collective oscillations in the cutaneous vascular system. Both types of motions, which lead to periodic changes in the optical signal, can be detected and measured by the apparatus 100 of this invention.

A set of periodic motions that is associated with cutaneous blood flow. The cardiac pulse rate is the rate at which the heart beats to pump blood into the circulatory system. The cardiac pulse rate is normally 1 Hz (one pulse per second). A second type of motion that is associated with a set of low frequency pulses, in the range from 0.1 Hz to 0.2 Hz, is dictated by the autonomous nervous system. A third periodic motion is the breathing motion, which matches the resting breathing frequency. The cutaneous circulatory system can be monitored to generate data 1o that are useful for diagnostic purposes. For example, vasomotion, the rhythmic contraction exhibited by the small arteries and arterioles, is reported to be impaired in diabetic subjects, relative to non-diabetic, healthy subjects (see K. B. Stansberry et al, “Impaired peripheral vasomotion in diabetes”, Diabetes Care 1996; Vol. 19: pages 715-721). The amplitude of the vasomotion becomes more prominent at lower temperatures, such as 22° C.

The method of this invention includes performing optical measurements of tissue and analyzing the optical measurements as a function of time, which measurements and analysis can be used to calculate concentration of hemoglobin, hematocrit value, cardiac pulse rate, blood pressure, and other vital signs. The method comprises the steps of:

a) collecting a set of optical measurements on a body part of a patient over a period of time;

b) determining at least one value of at least one blood parameter of the patient from the set of optical measurements, the blood parameter including, but not limited to, the concentration of hemoglobin or the hematocrit value of the patient;

c) determining at least one value of at least one vital sign of the patient from the set of optical measurements, the at least one vital sign including, but not limited to the cardiac pulse rate, blood pressure, and blood oxygen saturation;

d) reporting a combination of the at least one value of the at least one blood parameter and the at least one value of the at least one vital sign;

e) repeating steps a), b), c), and d) a sufficient number of times until a trend can be observed; and

f) activating an alarm when (1) the at least one value of the at least one vital sign crosses a specified cut-off value or (2) the rate of change in the at least one value of the at least one vital sign crosses a specified cut-off value or (3) the at least one value of the at least one blood parameter crosses a specified cut-off value or (4) the rate of change in the at least one value of the at least one blood parameter crosses a specified cut-off value.

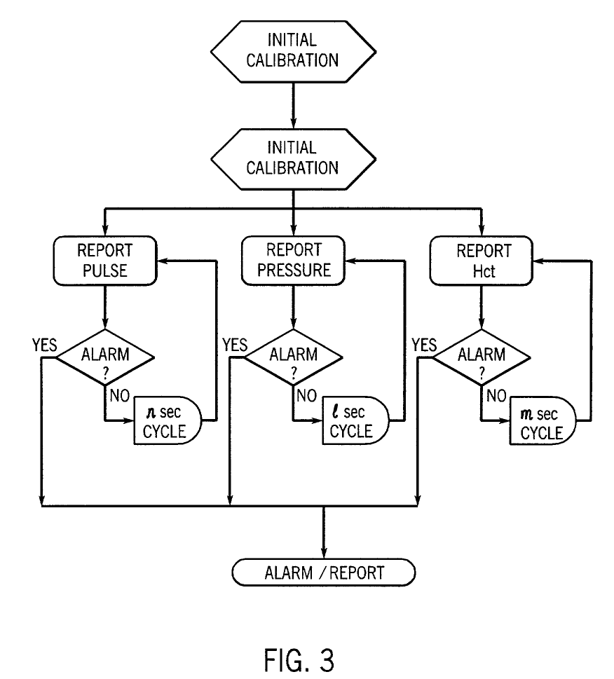

FIG. 3 is a flowchart depicting the steps for the determination of the hematocrit value and vital signs, or the change in the concentration of hemoglobin and vital signs, according to the method previously described. FIG. 3 also shows the preliminary steps of (1) initially calibrating the apparatus and (2) allowing the temperature of the body part to equilibrate.

The apparatus described in WO 99/59464 is capable of collecting data over an extended period of time (half a minute to few minutes), which data can be digitally filtered with the Fourier Transform algorithm to check the presence of periodic signals and determine the frequency and the amplitude of these signals. A plot can be constructed to show the amplitude of the periodic signal as a function of the frequency of this signal. If there is more than one periodic signal, the resultant plot is called the power spectrum. The power spectrum shows the relative magnitude of each periodic signal and the frequency range of each periodic signal.

It is possible to identify the cardiac pulse rate, cutaneous vasomotion, and respiratory motions from the power spectrum of the optical signal collected from an illuminated vascular system of a body part. Thus, in addition to the ability to determine the hematocrit value or the concentration of hemoglobin, a device substantially similar to that described in WO 99/59464 is capable of determining cutaneous periodic motions, including the cardiac pulse rate, cutaneous vasomotion, and respiration frequency.

The intensity of the light reflected or transmitted through tissue can be expressed by Beer's law, where OD=−log₁₀ I/Iₒ, where Iₒ represents the intensity of the light introduced into the tissue and I represents the intensity of the light reflected from or transmitted through the tissue.

A model of the measurement of the change in the concentration of hemoglobin that may be encountered by a patient during, for example, a period of hospitalization or as a result of injury, can be constructed. The concentration of hemoglobin is equal to the sum of the concentrations of its two components, oxygenated hemoglobin and deoxygenated hemoglobin. For a typical concentration of hemoglobin of 14.6 gm/dL, the optical density for a 1 cm path length measured with no applied pressure can be expressed as:

OD=ε_{HbO2}(1.6165×10⁻³)+ε_{RHb}(0.5489×10⁻³) (1)

where ε_{HbO2} represents the molar extinction coefficient in M⁻¹ cm⁻¹ for oxygenated hemoglobin and ERHb represents the molar extinction coefficient in M⁻¹ cm⁻¹ for deoxygenated hemoglobin, and the number 1.6165×10⁻³ is the molar concentration for oxygenated hemoglobin and the number 0.5489×10⁻³ is the molar concentration for deoxygenated hemoglobin, as calculated for a concentration of 14.6 gm/dL hemoglobin, with the assumption that oxygenated hemoglobin comprises 75% of total hemoglobin and deoxygenated hemoglobin comprises 25% of total hemoglobin.

The blood content of the tissue will change over a short period of time as a result of occlusion, bleeding, or hemodialysis. The change in the optical density from the initial concentration of hemoglobin (or the initial hematocrit value) to that at a subsequent value (ΔOD)ₜ, as a result of occlusion, bleeding, or hemodialysis, at any wavelength, can be expressed as:

Δ(OD)ₜ=ε_{HbO2}(Δ[HbO₂])ₜ+ε_{RHb}(Δ[RHb])ₜ (2)

where

Δ(OD)ₜ represents the difference in the measured optical density at a given wavelength and at time t,

ε_{HbO2} represents the molar extinction coefficient of oxygenated hemoglobin at the same wavelength,

Δ[HbO₂])ₜ represents the change in the concentration of oxygenated hemoglobin at time t,

ε_{RHb} represents the molar extinction coefficient of reduced hemoglobin (deoxygenated hemoglobin) at the same wavelength, and

(Δ[RHb])ₜ represents the change in the concentration of reduced hemoglobin (deoxygenated hemoglobin) at time t, wherein the change in concentration of hemoglobin results from occlusion, bleeding, or the effect of hemodialysis.

The coefficients in the expression are the values of extinction coefficients of oxygenated hemoglobin and deoxygenated hemoglobin and are available in the literature. These coefficients vary as a function of wavelength according to the following relationships:

$$\begin{matrix}{Δ ( OD )}_{t} at 590 nm = 14 \times {10}^{3} {( Δ [ {HbO}_{2} ] )}_{t} + 28 \times {10}^{3} {( Δ [ RHb ] )}_{t} & ( 3 ) \\ {Δ ( OD )}_{t} at 660 nm = 0.32 \times {10}^{3} {( Δ [ {HbO}_{2} ] )}_{t} + 3.2 \times {10}^{3} {( Δ [ RHb ] )}_{t} & ( 4 ) \\ {Δ ( OD )}_{t} at 735 nm = 0.41 \times {10}^{3} {( Δ [ {HbO}_{2} ] )}_{t} + 1.10 \times {10}^{3} {( Δ [ RHb ] )}_{t} & ( 5 ) \\ {Δ ( OD )}_{t} at 810 nm = 0.86 \times {10}^{3} {( Δ [ {HbO}_{2} ] )}_{t} + 0.72 \times {10}^{3} {( Δ [ RHb ] )}_{t} & ( 6 ) \\ {Δ ( OD )}_{t} at 890 nm = 1.2 \times {10}^{3} {( Δ [ {HbO}_{2} ] )}_{t} + 0.74 \times {10}^{3} {( Δ [ RHb ] )}_{t} & ( 7 ) \\ {Δ ( OD )}_{t} at 935 nm = 1.2 \times {10}^{3} {( Δ [ {HbO}_{2} ] )}_{t} + 0.73 \times {10}^{3} {( Δ [ RHb ] )}_{t} & ( 8 )\end{matrix}$$

The value of the change in the concentration of oxygenated hemoglobin (Δ[HbO₂]) and the value of the change in the concentration of deoxygenated hemoglobin (Δ[RHb]) can be obtained by solving any two of the foregoing equations, (3) through (8). The change in the concentration of total hemoglobin resulting from occlusion, bleeding, or changes during dialysis, can be determined by the equation:

Δ[Total Hb]ₜ=Δ[HbO₂]+Δ[RHb]ₜ (9)

An initial value of the concentration of total hemoglobin can be determined invasively, or non-invasively. The value of the concentration of total hemoglobin at time t, which may differ from the initial value (at time t=0) due to occlusion, bleeding, or because of changes during dialysis, is then calculated by using the equation:

[Total Hb]ₜ=Initial[Total Hb]±Δ[Total Hb]ₜ (10)

The Δ(OD)ₜ values, which are determined at several time intervals, starting from the onset of occlusion, the beginning of surgery, the beginning of post-operative care, or the beginning of a hemodialysis session, are used to calculate the change in concentration of total hemoglobin (±Δ[Total Hb]ₜ) resulting from occlusion, bleeding, or changes during dialysis by means of equation (9). The value of the concentration of total hemoglobin at the end of any other time interval, starting from the onset of occlusion, start of surgery, start of post-operative care, or start of a hemodialysis session, can then be determined by using equation (10).

It is possible to validate the method of calculating concentration of hemoglobin by performing a set of occlusion experiments. Occlusion of blood vessels in a body part involves application of pressure to the body part to limit the flow of blood from or if i to that part. The result of occlusion is a change in the amount of blood in the tissue under observation. Occlusion experiments can be used to illustrate the change in optical signal resulting from changes in blood content in the tissue. Occlusion can be considered as a substitute for changes in blood content, concentration of hemoglobin, or hematocrit value during surgery, post-operative care, or hemodialysis. For example, occluding a body part at a pressure above the value of the diastolic blood pressure and at a pressure below the value of systolic blood pressure will increase the concentration of oxygenated hemoglobin and the concentration of deoxygenated hemoglobin in the occluded tissue relative to the pre-occlusion values of these parameters. These increases are caused by the pooling of blood in the occluded tissue (e.g., the arm) as a result of closing the venous path that returns blood to the heart.

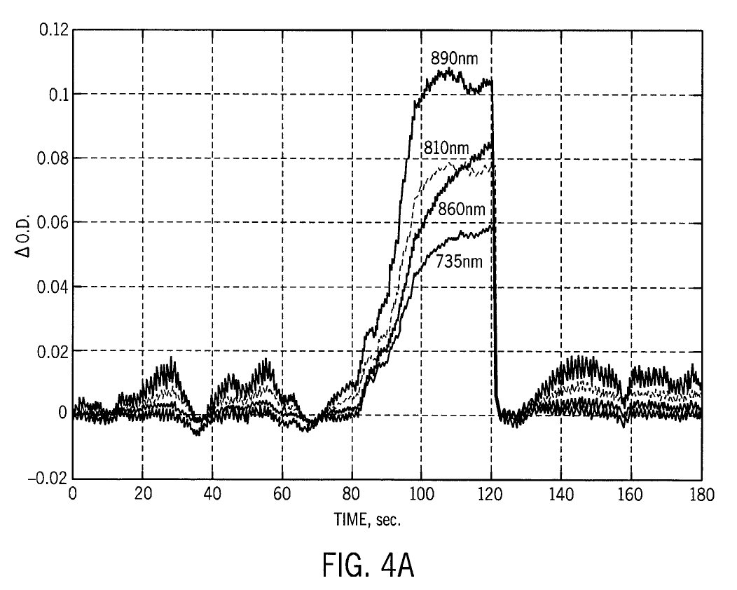

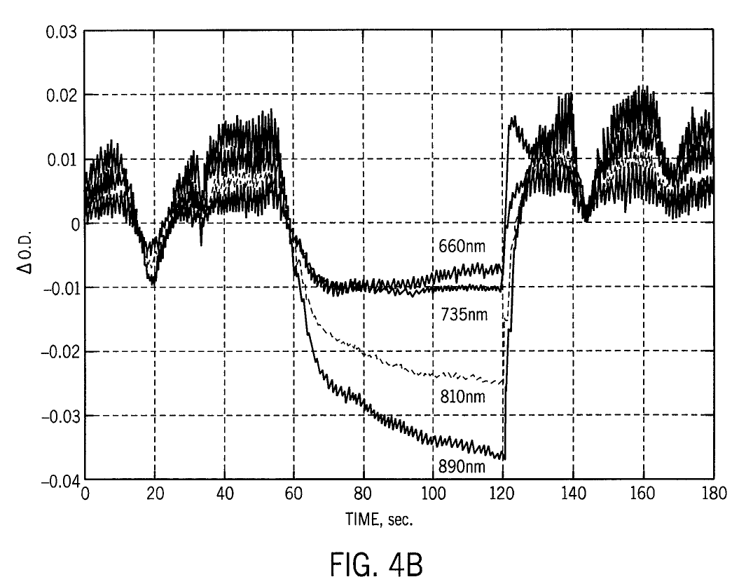

FIG. 4A shows the effect of occlusion on the optical signal under the following conditions: 130 mm Hg pressure, wavelengths of 660 nm, 735 nm, 810 nm, and 890 nm, light collected at a site at a distance of 1.86 mm from the light introduction site. FIG. 4B shows the effect of occlusion on the optical signal under the following conditions: 170 mm Hg pressure, wavelengths of 660 nm, 735 nm, 810 nm, and 890 nm, light collected at a site at a distance of 1.86 mm from the light introduction site. In this study, a blood pressure cuff was placed on the arm of a subject who was sitting in a clinical chair, the subject's left arm resting on the arm of the chair. The subject's index finger was placed in contact with the optical probe. The temperature in the aluminum disc was maintained at 38° C. The temperature of the finger was allowed to equilibrate with the disc for two minutes before measurements were begun. Data, i.e., optical signals, were collected for three minutes at the rate of 22 measurements of data per second. The data are presented as a plot of optical density (OD) vs. time in seconds. The pressure in the cuff was maintained at zero mm Hg for the first 60 seconds. The pressure was increased to 130 mm Hg, which was higher than the diastolic pressure and lower than the systolic pressure for the subject, and maintained at this pressure for 60 seconds. The pressure was released instantaneously, and data were collected for the remainder of the 180-second duration of the experiment. During the experiment the cardiac pulse rate, the oxygen saturation value, and a perfusion parameter were recorded by means of a Hewlett-Packard vital signs monitor having a plethysmographic sensor attached to the subject's middle finger. The measurement was repeated several times at different pressures, ranging from below the diastolic pressure to above the systolic pressure. The systolic pressure was defined as the pressure at which the pulse disappeared. For applied pressures below the systolic blood pressure, the back-flow of venous blood to the heart is stopped as a result of closing the venous path that returns blood to the heart, thus leading to a state of venous occlusion (FIG. 4A). The intensity of the reflected light decreased, i.e., the measured optical density increased (because pooled blood increases light absorption) until the optical density reached a plateau. As the pressure was reduced, the optical density returned to approximately the initial value of the optical density, i.e., the value prior to occlusion. When the arm of a subject is occluded at a pressure above the systolic blood pressure, arterial blood flow from the heart to the limb is stopped because of closing of the artery, and venous blood flow back to the heart is stopped because of closure of the return venous path, thus leading to a state of total ischemia, which is the state of total occlusion (FIG. 4B). The optical density measured at the finger decreases and reaches a plateau as a result of occlusion. As the pressure is released subsequent to total occlusion, the optical density increases (because pooling of blood increases light absorption) and returns to approximately its initial value, i.e., the value prior to occlusion. Similar response curves are observed at other wavelengths. It is possible to use equations (3) through (8) to calculate the change in the concentrations of oxygenated hemoglobin and deoxygenated hemoglobin and hence, the change in concentration of total hemoglobin at the finger when the arm is occluded.

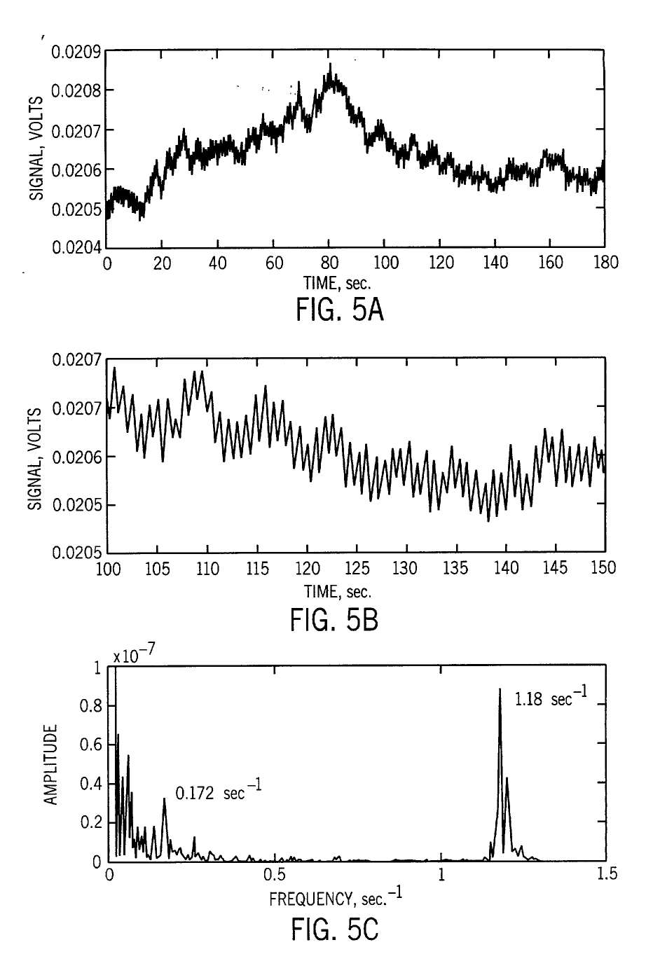

The cardiac pulse rate can be determined from the optical signals collected from a body part. The optical signals collected from a human body part over a period of time is a composite of several periodic signals that includes signals arising from the cardiac pulse rate, breathing rate, and vasomotion, as shown in FIG. 5A. FIG. 5A is a graph showing the intensity of the reflected light from the forearm of a human subject at 590 nm and at a sampling distance of 1.86 mm as a function of time. Signals were collected over a three-minute period. The temperature of the skin was maintained at 41° C. FIG. 5B is a graph showing a the portion of FIG. 5A from the point of time of 100 seconds to the point of time of 150 seconds. FIG. 5C is a plot of the calculated Fourier Transform of the amplitude of the reflected light signal shown in FIG. 5A.

The cardiac pulse rate can be determined by recording the output of the optical probe over several pulses, over a given period of time. By expanding a portion of FIG. 5A (see FIG. 5B), it can be seen that the cardiac pulse rate is superimposed over other pulses having lower frequency. By performing a Fourier Transform, a plot of the power spectrum can be constructed, which plot shows the cardiac pulse rate at 1.18 Hz (see FIG. 5C) and several low frequency pulses indicative of other oscillations in the vascular system of the skin.

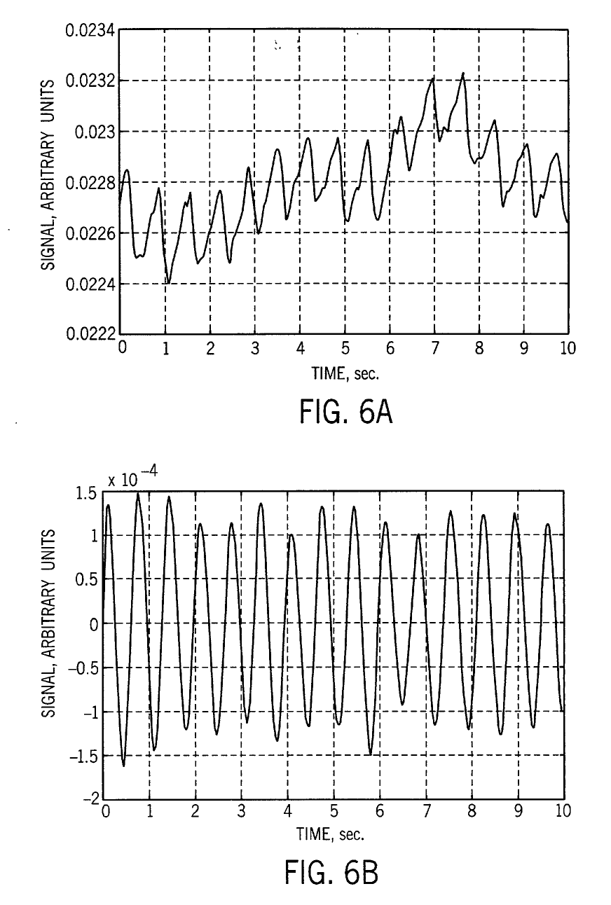

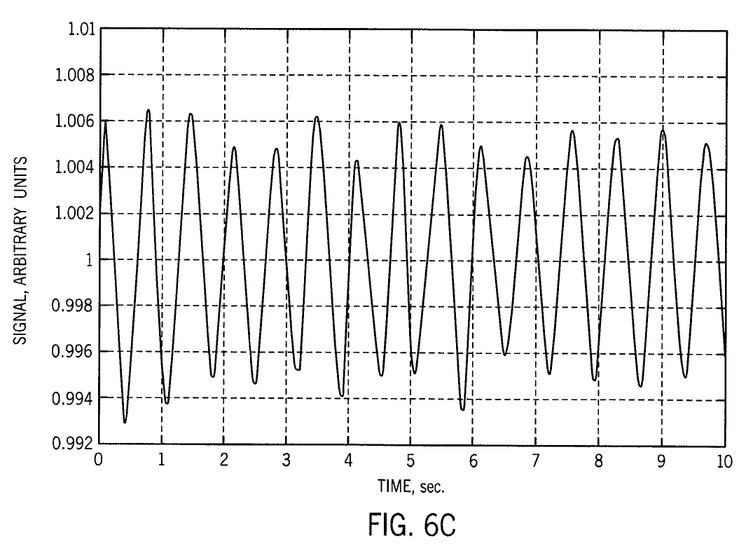

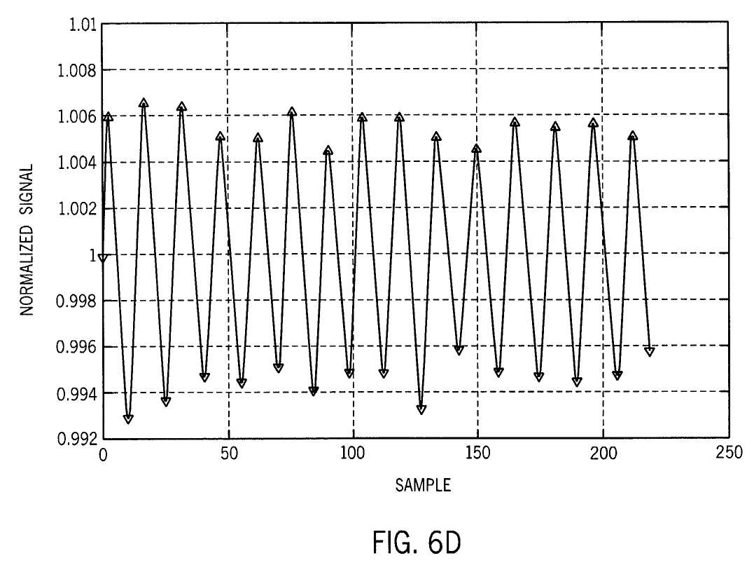

Alternatively, the cardiac pulse rate can be calculated from the filtered signal. See FIG. 6C. The cardiac pulse rate can be reported as pulses per second by counting the number of peaks or valleys of the filtered pulses over a period of time and calculating the cardiac pulse rate in pulses per minute. This calculation is shown in Example 2.

Arterial oxygen saturation can be determined by (a) calculating the changes in optical density for a set of digitally filtered pulses at more than two wavelengths, (b) calculating the concentrations of oxygenated hemoglobin and deoxygenated hemoglobin from these measurements, and (c) then deriving the value of oxygen saturation, expressed as a percentage.

The value of arterial oxygen saturation can be calculated from the output of the optical probe at two wavelengths, and with no occlusion pressure being applied. The method for determination of oxygen saturation by using the apparatus of this invention comprises the steps of:

1) collecting optical signals for a plurality of pulses, the optical signals being generated from light at a first wavelength;

2) digitally filtering the signals collected to reject the low frequency pulses, which are possibly associated with breathing frequency and vasomotion, and high frequency noise, which is possibly associated with electronic noise;

3) normalizing the digitally filtered signal of each pulse to the mean value of the signals collected over the period of measurement;

4) locating the peak and the valley of each digitally filtered pulse;

5) determining the intensity of the signal at each peak (Iₚ) and at each valley (Iᵥ) of each digitally filtered pulse, where

Iₚ represents the intensity of the signal at the peak of a cardiac pulse wave, and

Iᵥ represents the intensity of the signal at the valley of a cardiac pulse wave;

6) determining the average value of the peak intensities and the average value of the valley intensities of each digitally filtered pulse;

7) determining the value of the logarithm of the peak intensity for each pulse and the value of the logarithm of the valley intensity for each pulse to provide a value of optical density for each pulse, where

(OD)ᵥ=−log (Iᵥ/Iₒ)

(OD)ₚ=−log (Iₚ/Iₒ)

8) calculating the difference in the average optical density for each pulse,

where

Δ(OD) during pulse=(OD)ₚ−(OD)ᵥ;

9) repeating steps 1) through 8), wherein the optical signals are generated from light at a second wavelength, the second wavelength not being the same as the first wavelength;

10) calculating the concentration of oxygenated hemoglobin ([HbO₂]) and the concentration of deoxygenated hemoglobin ([RHb]) by means of simultaneous equations, where

Δ(OD)_{λ1}=a[HbO₂]+b[RHb]

Δ(OD)_{λ2}=c[HbO₂]+d[RHb]

and the coefficients a, b, c, and d vary for each pair of wavelengths according to the values in Table 1; and

11) calculating the value of oxygen saturation according to the following equation:

$$Arterial oxygen saturation = \frac{[ {HbO}_{2} ]}{( [ {HbO}_{2} ] + [ RHb ] )} \times 100 \%$$

The coefficients a, b, c, and d in step 10) are the values of the extinction coefficients at the maximum wavelength of the particular LED. The approximation does not take into consideration the finite bandwidth of the LED or the skew of the intensity distribution over the bandwidth.

TABLE 1

| | Linear coefficients (values of the extinction | | | |
|---|---|---|---|---|
| | coefficients at the maximum wavelength of each LED) | | | |
| Wavelength pair | a | b | c | d |
| 735 nm/ | 410 | 1100 | 860 | 720 |
| 810 nm | | | | |
| 735 nm/ | 410 | 1100 | 1178 | 744 |
| 890 nm | | | | |
| 660 nm/ | 320 | 3223 | 860 | 720 |
| 810 nm | | | | |
| 660 nm/ | 320 | 3223 | 1178 | 730 |
| 890 nm | | | | |

Blood pressure can be measured by placing a pressure cuff around the arm and inflating the cuff while a stethoscope is placed over the brachial artery in the arm and under the cuff. When the pressure is equal to or higher than the systolic pressure, arterial occlusion occurs, and the stethoscope will detect no pulses. The pressure induced by the cuff is slowly reduced, and the systolic pressure is the value of the pressure at which the cardiac pulse signal is first detected by the stethoscope. The pressure induced by the cuff is gradually lowered an additional amount, and the diastolic pressure is subsequently determined to be the pressure at which the audible pulse signal vanishes.

Alternatively, an optical signal generated from and collected by an optical probe in contact with a body part where the blood pressure measurement is taken can be used to determine the blood pressure. In this case, the systolic blood pressure is the pressure at which a regular (periodic) pulse rate disappears. The optical signal is a function of pressure in the cuff applied to the body part.

As another alternative, the systolic blood pressure can be measured by determining the frequency of the low frequency vasomotion at a constant temperature, after the respiratory frequency is separated from the vasomotion frequency. The systolic blood pressure can be calculated from the amplitude of the low frequency pulses at a constant temperature.

The apparatus of this invention comprises an integrated structure comprising an optical probe, the probe capable of performing optical measurements of tissue, which measurements are used to calculate the concentration of hemoglobin, the hematocrit value, the cardiac pulse rate, blood pressure, and other vital signs. The apparatus of this invention can also monitor changes in the hematocrit value and vital signs for patients who are at high risk of postoperative complications. The method of this invention can be used to monitor changes in blood parameters and change in vital signs of a patient during postoperative care or while the patient is in an intensive care unit to detect internal bleeding. It is also possible to measure the response of human body parts (including skin) to changes in temperature and occlusion pressure at different wavelengths by means of the optical probe described herein.

### EXAMPLES

### Example 1

### Use of Apparatus

Referring now to FIGS. 2A, 2B, and 2C, an optical probe suitable for carrying out the method of this invention comprises a set of light emitting diodes (LEDs) that emit light at wavelengths 590 nm, 660 nm, 890 nm, and 935 nm. The output of the LEDs is focused on a light introduction fiber 120 that transmits light from the LEDs to the skin at a light introduction site. Each light emitting diode (LED) can be operated in a modulated current mode by modulating the current input to each LED at a fixed frequency. Alternatively, LEDs can be operated in a constant current mode.

In this example, LED 1 emits light having a wavelength of 660 nm, a modulation frequency of 1024 Hz, and a half bandwidth of 15 nm. LED 2 emits light having a wavelength of 590 nm, a modulation frequency of 819 Hz and a half bandwidth of 15 nm. LED 3 emits light having a wavelength of 935 nm, a modulation frequency of 585 Hz, and a half bandwidth of 25 nm. LED 4 emits light having a wavelength of 890 nm, a modulation frequency of 455 Hz, and a half bandwidth of 25 nm.

Light from each of the four LEDs was introduced into the body part by means of a light introduction fiber (silica, 0.4 mm in diameter) and light re-emitted from the body part was collected by four light collection fibers (silica, 0.4 mm in diameter). The centers of the four light collection fibers were placed at distances of 0.44 mm, 0.92 mm, 1.21 mm, and 1.84 mm from the center of the light introduction fiber. Light collected was detected by silicon photodiodes, the signals were amplified, and the resultant amplified signals were digitized by means of an analog to digital converter board (National Instruments, Austin, Tex.) and processed by a personal computer.

The signals were collected by placing the optical probe in contact with a finger of the subject. Each signal collected at each separation of the light introduction site from the light collection site was a composite of the intensities of reflected light at four wavelengths, each signal modulated at a different frequency. The Fourier Transform algorithm was applied to the signal at each detector (corresponding to each separation of the light introduction site from the light collection site) to provide the intensity of the reflected light at each separation of the light introduction site from the light collection site and at each specified wavelength.

### Example 2

### Cardiac Pulse Rate

The optical probe 116 located in the lower part 102 of the apparatus 100 illustrated in FIG. 2A was used in different set-ups to illustrate the ability of the apparatus to perform cardiac pulse rate measurements, arterial blood oxygen saturation measurement, and response of cutaneous blood vessels to occlusion.

FIGS. 5A, 5B, 5C, and 5D show the results of the steps carried out to calculate the cardiac pulse rate from optical signals. These steps were as follows:

1) collecting optical signals for a plurality of pulses, the optical signals being generated from light at a first wavelength;

2) digitally filtering the signals collected to reject the low frequency pulses, which are possibly associated with breathing frequency and vasomotion, and high frequency noise, which is possibly associated with electronic noise;

3) normalizing the digitally filtered signal of each pulse to the mean value of the signals collected over the period of measurement;

4) locating the peak and the valley of each digitally filtered pulse; and

5) calculating the cardiac pulse rate by determining the number of peaks per minute.

The cardiac pulse rate was calculated from the first section of the curve at zero occlusion, at both 38° C. and 22° C. The data for a subject with normal perfusion condition are shown in Table 2 (38° C.) and in Table 3 (22° C.).

TABLE 2

| | Cardiac pulse rate | Cardiac pulse rate | Cardiac pulse rate | Cardiac pulse rate |
|---|---|---|---|---|
| | measured at 0.44 | measured at 0.92 | measured at 1.21 | measured at 1.78 |
| Wavelength (nm) | mm | mm | mm | mm |
| 660 | 73 | 73 | 73 | 73 |
| 735 | 73 | 73 | 73 | 73 |
| 810 | 73 | 73 | 73 | 73 |
| 890 | 73 | 73 | 73 | 73 |

The average cardiac pulse rate at all separations of the light introduction site from the light collection site and at all wavelengths was 73 pulses per minute at 38° C.

TABLE 3

| | Cardiac pulse rate | Cardiac pulse rate | Cardiac pulse rate | Cardiac pulse rate |
|---|---|---|---|---|
| | measured at 0.44 | measured at 0.92 | measured at 1.21 | measured at 1.78 |
| Wavelength (nm) | mm | mm | mm | mm |
| 660 | 69 | 69 | 73 | 69 |
| 735 | 69 | 69 | 69 | 69 |
| 810 | 69 | 69 | 69 | 69 |
| 890 | 73 | 73 | 69 | 73 |

The average cardiac pulse rate at all separations of the light introduction site from the light selection site and at all wavelengths was 73 pulses per minute at 22° C.

The cardiac pulse rate was also checked by a reference clinical instrument, Hewlett-Packard vital signs monitor, which had a plug-in bay (model no. M1046) and a calculation and display unit (model no. M1092 M). Several plug-in modules were used. These included the oxygen saturation module (model no. M1020A) and the blood pressure module (model no. M1008B). An optical probe for determining oxygen saturation was placed in contact with the subject's finger and connected to the oxygen saturation module. A blood pressure cuff was placed on the subject's arm and the signals from the attached sensor were input to the Hewlett-Packard blood pressure module. The reference device was used for measuring cardiac pulse rate, oxygen saturation, and blood pressure.

The value of the cardiac pulse rate measured on the patient clinical monitor (Hewlett-Packard vital sign monitor) that was used as a reference ranged from 68 to 73 pulses per minute during the study. One of the light sources used with the Hewlett-Packard vital signs monitor was the 660 nm LED and light was transmitted through the entire digit of the finger.

### Example 3

### Oxygen Saturation

The value of oxygen saturation can be calculated from the signals generated and collected by the optical probe measured at two wavelengths and with no occlusion pressure applied.

The determination of oxygen saturation by the method of this invention comprised the steps of:

1) collecting optical signals for a plurality of pulses, the optical signals being generated from light at a first wavelength;

2) digitally filtering the signals collected to reject the low frequency pulses, which are possibly associated with breathing frequency and vasomotion, and high frequency noise, which is possibly associated with electronic noise;

3) normalizing the digitally filtered signal of each pulse to the mean value of the signals collected over the period of measurement;

4) locating the peak and the valley of each digitally filtered pulse;

5) determining the intensity of the signal at each peak (Iₚ) and at each valley (Iᵥ) of each digitally filtered pulse, where

Iₚ represents the intensity of the signal at the peak of a cardiac pulse wave, and

Iᵥ represents the intensity of the signal at the valley of a cardiac pulse wave;

6) determining the average value of the peak intensities and the average value of the valley intensities of each digitally filtered pulse;

7) determining the value of the logarithm of the peak intensity for each pulse and the value of the logarithm of the valley intensity for each pulse to provide a value of optical density for each pulse, where

(OD)ᵥ=−log (Iᵥ/Iₒ)

(OD)ₚ=−log (Iₚ/Iₒ)

8) calculating the difference in the average optical density for each pulse, where

Δ(OD) during pulse=(OD)ₚ−(OD)v;

9) repeating steps 1) through 8), wherein the optical signals are generated from light at a second wavelength, the second wavelength not being the same as the first wavelength;

10) calculating the concentration of oxygenated hemoglobin ([HbO₂]) and the concentration of deoxygenated hemoglobin ([RHb]) by means of simultaneous equations, where

Δ(OD)_{λ1}=a[HbO₂]+b[RHb]

Δ(OD)_{λ2}=c[HbO₂]+d[RHb]

and the coefficients a, b, c, and d vary for each pair of wavelengths according to the values in Table 1; and

11) calculating the value of oxygen saturation according to the following equation:

$$Arterial oxygen saturation = \frac{[ {HbO}_{2} ]}{( [ {HbO}_{2} ] + [ RHb ] )} \times 100 \%$$

The coefficients a, b, c, and d in step 10) are the values of the extinction coefficients at the maximum wavelength of the particular LED. The approximation does not take into consideration the finite bandwidth of the LED or the skew of the intensity distribution over the bandwidth.

The wavelength pairs 660 nm/810 nm and 660 nm/890 nm yielded oxygen saturation values between 91 and 100 at all separations of the light introduction site from the light collection site. The calculated oxygen saturation (O₂ sat) values for a normal subject are shown in Table 4.

TABLE 4

| | O₂ sat at 0.44 mm | O₂ sat 0.92 mm | O₂ sat 1.21 mm | O₂ sat 1.78 mm |
|---|---|---|---|---|
| 660 nm/810 nm | | | | |
| 38° C. | 93 | 94 | 93 | 91 |
| 22° C. | 93 | 89 | 92 | 87 |
| 660 nm/890 nm | | | | |
| 38° C. | 93 | 98 | 95 | 93 |
| 22° C. | 94 | 91 | 95 | 85 |

The average measured value of oxygen saturation at all separations of the light introduction site from the light collection site with the 660 nm/810 nm pair was 92.5% at 38° C. and 90% at 22° C. The average measured value of oxygen saturation at all separations of the light introduction site from the light collection site with the 660 nm/890 nm pair was 94.75% at 38° C. and 91.25% at 22° C.

The oxygen saturation value measured by the Hewlett-Packard vital signs monitor that was used as a reference was in the range of 92% to 95% during the experiment. One of the light sources used with the Hewlett-Packard vital signs monitor was the 660 nm LED and light was transmitted through the entire digit of the finger.

### Example 4

### Change in Concentration of Hemoglobin

The initial hematocrit value is determined either by an invasive method or by a non-invasive method. The optical density at the measurement site is determined at the time the concentration of hemoglobin or the hematocrit value is measured by contacting the optical probe with the body part. The optical density of the tissue of the finger (OD) is determined at the wavelengths 660 nm, 735 nm, 810 nm, and 890 nm at another time t, after the initial measurement. At least two of the following four linear equations are solved to obtain the values of A[HbO₂] and A[RHb].

$$\begin{matrix}{Δ ( OD )}_{t} at 660 nm = 0.32 \times {10}^{3} {( Δ [ {HbO}_{2} ] )}_{t} + 3.2 \times {10}^{3} {( Δ [ RHb ] )}_{t} & ( 4 ) \\ {Δ ( OD )}_{t} at 735 nm = 0.41 \times {10}^{3} {( Δ [ {HbO}_{2} ] )}_{t} + 1.10 \times {10}^{3} {( Δ [ RHb ] )}_{t} & ( 5 ) \\ {Δ ( OD )}_{t} at 810 nm = 0.86 \times {10}^{3} {( Δ [ {HbO}_{2} ] )}_{t} + 0.72 \times {10}^{3} {( Δ [ RHb ] )}_{t} & ( 6 ) \\ {Δ ( OD )}_{t} at 890 nm = 1.2 \times {10}^{3} {( Δ [ {HbO}_{2} ] )}_{t} + 0.74 \times {10}^{3} {( Δ [ RHb ] )}_{t} & ( 7 )\end{matrix}$$

The change in concentration of total hemoglobin can be determined by the equation:

Δ[Total Hb]=Δ[HbO₂]+Δ[RHb] (9)

The value of the concentration of total hemoglobin is then updated to the new value by means of the equation:

[Total Hb]=Initial[Total Hb]±Δ[Total Hb] (10)

Either the value of the concentration of total hemoglobin is reported or the change in the hematocrit value is reported. If the change in the concentration of total hemoglobin is negligible or slightly positive, then no alarm is activated. However, if the change in concentration of total hemoglobin is negative and crosses a specified cut-off value, then an alarm can be activated.

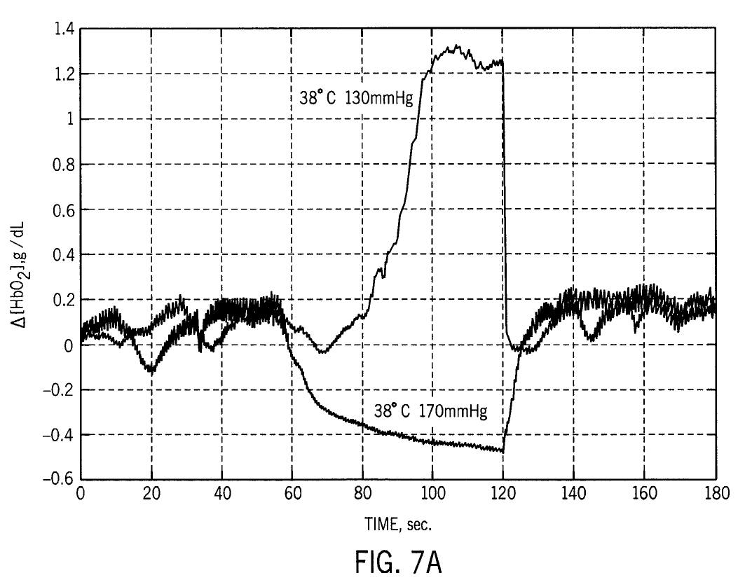

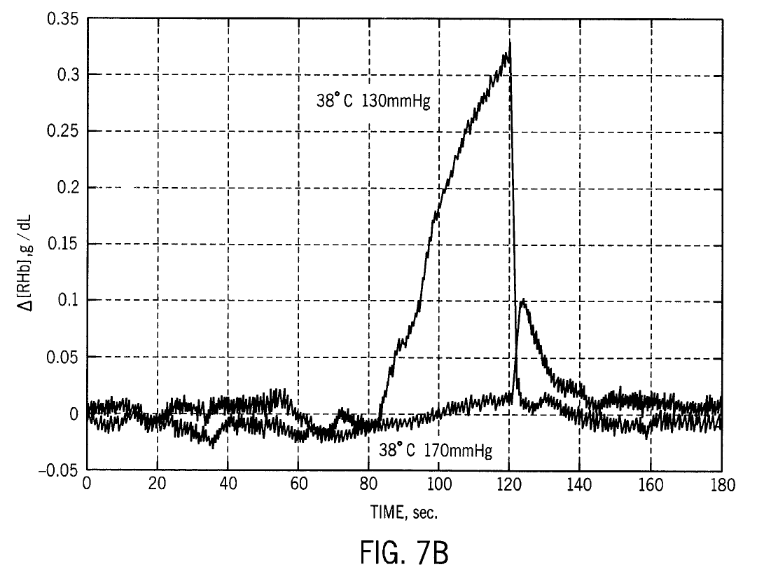

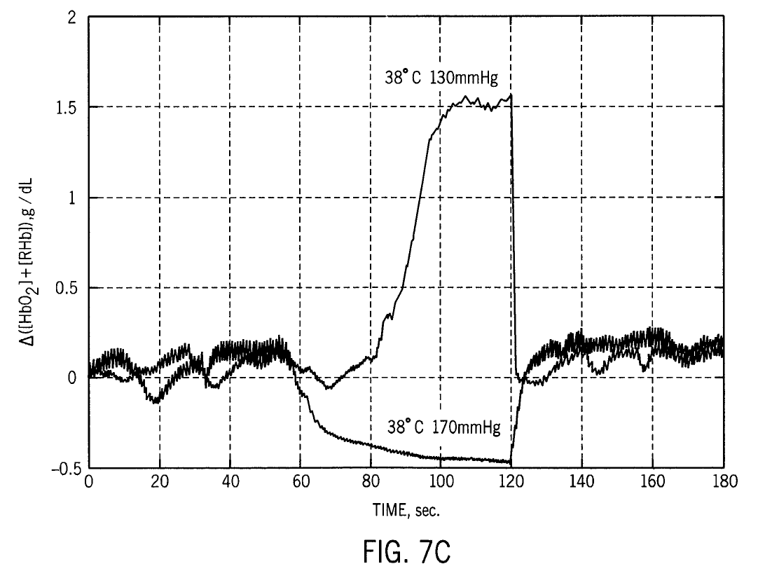

FIG. 7A and FIG. 7B show the change in concentration of oxygenated hemoglobin and the change in concentration of deoxygenated hemoglobin as a result of venous or arterial occlusion. The calculated change in concentration of hemoglobin is shown in FIG. 7C.

### Example 5

### Hemoglobin and Hematocrit

The concentration of hemoglobin and the hematocrit value can be determined by means of the method of this invention by means of an apparatus substantially similar to that described in WO 99/59464, which is substantially similar to the apparatus of this invention. In this method a calibration relationship is established for a population of size sufficient to encompass the skin color and hematocrit range for the patient to be monitored. The initial concentration of hemoglobin or the initial hematocrit value is determined from an optical measurement and the established calibration relationship. This calibration relationship is established by collecting data both non-invasively and invasively and applying a fitting algorithm, such as linear least squares or partial least squares, to the data to determine coefficients of a linear equation, standard error of prediction, and correlation coefficient. Once an initial value of the concentration of hemoglobin or the initial hematocrit value is established, the apparatus of this invention is brought into continuous contact with the body part of the patient and the latest signal and the corresponding concentration of hemoglobin and hematocrit value are recorded.

A study was conducted to illustrate the ability of the method of this invention to track changes in the hematocrit value caused by bleeding. Four healthy subjects volunteered to donate blood. The hematocrit value for the blood of these subjects was determined prior to donation. Non-invasive measurements were then taken with light having wavelengths of 590 nm, 650 nm, 750 nm, 800 nm, 900 nm, and 950 nm by means of the breadboard optical sensor described in WO 99/59464, but employing a tungsten light source and a set of filters to select the wavelengths. Optical signals were collected at the six wavelengths and at six separations of the light introduction site form the light collection site. The temperature of the skin was maintained at 34° C. Each of the four subjects was tested five times over a seven-day period. The data were used to generate a calibration set, and a four-term regression model was used to generate the calibration and cross-validation relationships. The calibration model contained data collected over the seven-day period, before and after the donation of one pint of blood (473 ml). Performance was judged by the calibration coefficient, R(calibration), which was 0.96, the standard error of calibration, SE(calibration), which was 1.11 HCt units, leave-one-out cross validation coefficient, R(cross validation), which was 0.94, and the standard error of cross validation prediction, SE(cross validation), which was 1.22 Hct units. The hematocrit determination and the non-invasive measurement were also performed two weeks after the end of the seven-day period. The predicted hematocrit values for three of the four subjects correlated with the values determined invasively with a correlation coefficient R=0.98. Thus, the apparatus and method of this invention were capable of tracking the change in the hematocrit value as a result of bleeding (donating one pint of blood) over a period of time.

### Example 6

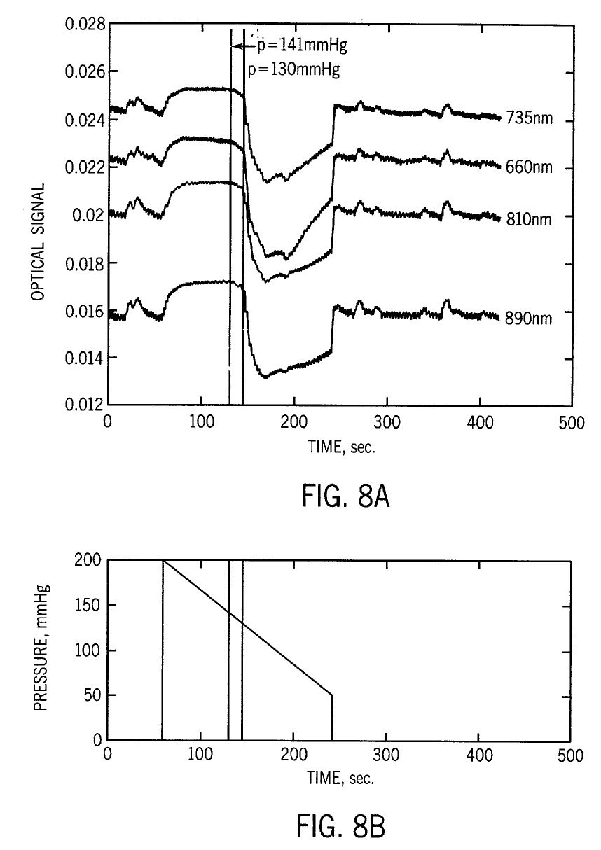

The optical probe of this invention is capable of montoring the systolic blood pressure of a patient and the change in blood pressure as a function of time. FIG. 8A shows a tracing of the optical signal versus time for a human finger as the pressure in a cuff was rapidly increased to 200 mm Hg and slowly decreased to 50 mm Hg over a period of 180 seconds. Pressure was applied to the left arm at the 60-seconds point in time to bring about occlusion. The cuff pressure was increased from zero to 200 mm Hg within approximately two seconds. The pressure was then slowly reduced. A plot of the cuff pressure versus time is shown in FIG. 8B. The rate of pressure reduction was 0.833 mm Hg per second. Upon occlusion of the blood vessels in the left arm, the optical signal increased and remained at a plateau until a pressure of 141 mm Hg was reached and then sharply decreased as the pressure fell below 130 mm Hg. The blood pressure of the subject was measured on the right arm immediately before the study, and the systolic pressure was 134±5 mm Hg. Thus, the inflection point in the plot of optical signal versus time (deflation time) lies at the systolic blood pressure of the subject. Accordingly, it is possible to track the systolic blood pressure of a person using the apparatus and method of this invention.

Various modifications and alterations of this invention will become apparent to those skilled in the art without departing from the scope and spirit of this invention, and it should be understood that this invention is not to be unduly limited to the illustrative embodiments set forth herein.

What is claimed is:

1. An apparatus for monitoring changes in blood parameters and vital signs of a patient, said apparatus comprising:
a) means for illuminating a body part of a patient;
b) means for collecting optical signals over a period of time from said body part;
c) means for effecting pressure changes or temperature changes or both of the foregoing types of changes in said body part;
d) means for measuring pressure changes or temperature changes or both types of the foregoing changes experienced by said body part;
e) means for calculating at least one value of at least one blood parameter of said patient from the collected optical signals;
f) means for determining at least one value of at least one vital sign of the patient from said collected optical signals;
g) means for reporting said at least one value of said at least one blood parameter and said at least one value of said at least one vital sign; and
h) means for providing an alarm when (1) said at least one value of said at least one vital sign crosses a specified cut-off value or (2) the rate of change in said at least one value of said at least one vital sign crosses a specified cut-off value or (3) said at least one value of said at least one blood parameter crosses a specified cut-off value or (4) the rate of change in the at least one value of the at least one blood parameter crosses a specified cut-off value.

2. The apparatus of claim 1, wherein said at least one vital sign is selected from the group consisting of cardiac pulse rate, temperature, oxygen saturation, blood pressure, and respiratory rate.

3. The apparatus of claim 1, wherein said means of illuminating said body part and collecting optical signal from said body part is attachable to said body part.

4. The apparatus of claim 1, wherein the temperature of said means of illuminating said body part and collecting optical signal from said body part is controllable.

5. The apparatus of claim 1, wherein said means of illuminating said body part and collecting optical signal from said body part employs light having wavelengths in the range of from about 500 nm to about 2000 nm.

6. The apparatus of claim 1, wherein said means of illuminating said body part and collecting optical signal from said body part employs light having wavelengths in the range of from about 500 nm to about 1100 nm.

7. A method for monitoring at least one blood parameter and at least one vital sign of a patient, said method comprising the steps of:
a) collecting a set of optical measurements on a body part of a patient over a period of time;
b) determining at least one value of at least one blood parameter of said patient;
c) determining at least one value of at least one vital sign of said patient from said set of optical measurements;
d) reporting a combination of said at least one value of said at least one blood parameter and said at least one value of said at least one vital sign;
e) repeating steps a), b), c), and d) a sufficient number of times until a trend can be observed; and
f) activating an alarm when (1) said at least one value of said at least one vital sign crosses a specified cut-off value or (2) the rate of change in said at least one value of said at least one vital sign crosses a specified cut-off value or (3) said at least one value of said at least one blood parameter crosses a specified cut-off value or (4) the rate of change in said at least one value of said at least one blood parameter crosses a specified cut-off value.

8. The method of claim 7, wherein said optical measurements are performed while said body part is subjected to more than one occlusion condition.

9. The method of claim 7, wherein said optical measurements are performed periodically in accordance with a preset program.

10. The method of claim 7, wherein said optical measurements are synchronized with the application of a blood pressure cuff applied upstream from the location of the optical measurement.

11. The method of claim 7, wherein said at least one vital sign is selected from the group consisting of cardiac pulse rate, a blood pressure parameter. and an oxygen saturation parameter.

12. The method of claim 7, wherein the concentration of hemoglobin or the hematocrit value is determined at an initial time invasively.

13. The method of claim 7, wherein the concentration of hemoglobin or the hematocrit value is determined at an initial time non-invasively.

14. The method of claim 7, wherein said initial concentration of hemoglobin is determined in said body part non-invasively using light having wavelengths in the range of from about 500 nm to about 2000 nm.

15. The method of claim 14, wherein said initial concentration of hemoglobin is determined in said body part non-invasively using light having wavelengths in the range of from about 500 nm to about 1100 nm.

16. The method of claim 14, wherein said concentration of hemoglobin is periodically updated by adding said change in concentration of hemoglobin to said initially determined concentration.

17. The method of claim 7, wherein said blood pressure measuring steps utilizes an optical sensor that is integrated with said hematocrit and cardiac-pulse sensor.

18. The method of claim 7, wherein said blood pressure measuring sensor is an optical sensor independent of but integrated with said hematocrit and cardiac-pulse rate sensor.

19. The method of claim 7, wherein said optical measurement is performed at constant temperature.
